(19) United States
(12) Patent Application Publication
(10) Pub. No.: US 2012/0237468 A1
(43) Pub. Date: Sep. 20, 2012

(54) CONDITIONING COMPOSITION COMPRISING AMIDOAMINE AND LACTIC ACID

(75) Inventors: Peter Lawrence Bailey, Bebington (GB); Stephen Leonard Briggs, Bebington (GB); Kayo Kobori, Bebington (GB)

(73) Assignee: Unilever PLC, London (GB)

(21) Appl. No.: 13/502,202

(22) PCT Filed: Oct. 4, 2010
(86) PCT No.: PCT/EP10/64719

§ 371 Date: May 18, 2012

(30) Foreign Application Priority Data
Oct. 21, 2009 (EP) 09173618.1
Nov. 16, 2009 (EP) 09176027.2

(51) Int. Cl. A61K 8/40; A61Q 5/12

(52) U.S. Cl. 424/70.28

(57) ABSTRACT

Conditioning composition comprising a quaternary conditioning surfactant, an acid neutralized amidoamine surfactant of general formula: R1-C(O)-NH-R2-N(R3)(R4) wherein R1 is a fatty acid chain with from 12 to 22 carbon atoms, R2 is an alkylene group containing from one to 4 carbon atoms and R3 and R4 are, independently, an alkyl group having from one to four carbon atoms and from 0.45 to 4% wt. of the composition lactic acid.

The present invention relates to an improved conditioning composition.

Despite the prior art there remains the need for improved conditioning compositions.

Accordingly, the present invention provides a conditioning composition comprising a quaternary conditioning surfactant, an acid neutralized amidoamine surfactant of general formula:
R1-C(O)-NH-R2-N(R3)(R4)
   wherein R1 is a fatty acid chain with from 12 to 22 carbon atoms, R2 is an alkylene group containing from one to 4 carbon atoms and R3 and R4 are, independently, an alkyl group having from one to four carbon atoms and from 0.45 to 4% wt. of the composition lactic acid.

Surprisingly, it has been found that the claimed system provides significant hair fibre restructuring benefits without having an excessively low pH, despite the level of acid.

Preferably, the lactic acid is present at from 0.5 to 3.5% wt. of the composition and more preferably at from 0.6 to 2% wt. of the composition.

The composition according to the invention will also comprise one or more conditioning surfactants which are cosmetically acceptable and suitable for topical application to the hair.

Suitable conditioning surfactants are selected from cationic surfactants, used singly or in admixture. Examples include quaternary ammonium hydroxides or salts thereof, e.g. chlorides.

Suitable cationic surfactants for use in hair conditioners of the invention include cetyltrimethylammonium chloride, behenyltrimethylammonium chloride, cetylpyridinium chloride, tetramethylammonium chloride, tetraethylammonium chloride, octyltrimethylammonium chloride, dodecyltrimethylammonium chloride, hexadecyltrimethylammonium chloride, octyldimethylbenzylammonium chloride, decyldimethylbenzylammonium chloride, stearyldimethylbenzylammonium chloride, didodecyldimethylammonium chloride, dioctadecyldimethylammonium chloride, tallowtrimethylammonium chloride, cocotrimethylammonium chloride, and the corresponding hydroxides thereof. Further suitable cationic surfactants include those materials having the CTFA designations Quaternium-5, Quaternium-31 and Quaternium-18. Mixtures of any of the foregoing materials may also be suitable. Particularly useful cationic surfactants for use in hair conditioners of the invention are cetyltrimethylammonium chloride and behenyltrimethylammonium chloride, available commercially, for example as DEHYQUART, ex Henkel.

The most preferred conditioning surfactants are cetyl trimonium chloride and behenyl trimonium chloride.

The level of conditioning surfactant is preferably from 0.01 to 10%, more preferably 0.05 to 5%, most preferably 0.1 to 3.5% by weight of the composition.

The pH of the composition is from 2.0 to 4.0, more preferably from 3.1 to 3.8.

The acid neutralized amidoamine surfactant is a compound of general formula:
R1-C(O)-NH-R2-N(R3)(R4)
   wherein R1 is a fatty acid chain with from 12 to 22 carbon atoms, R2 is an alkylene group containing from one to 4 carbon atoms and R3 and R4 are, independently, an alkyl group having from one to four carbon atoms.

Preferably, the acid neutralized amidoamine surfactant is selected from stearamidopropyl dimethylamine, stearamidopropyl diethylamine, stearamidoethyl dimethylamine, stearamidoethyl diethylamine, palimtamidopropyl dimethylamine, behenamidopropyl dimethylamine, myristamidopropyl dimethylamine, oleoamidopropyl dimethylamine, ricinoleoamidopropyl dimethylamine and mixtures.

Preferably, the amidoamine is present at from 0.1 to 10% wt. of the composition, more preferably from 0.1 to 5% wt and most preferably from 0.1 to 2% wt. of the composition.

Suitable basic amino acids include arginine, lysine and histidine may also be used as buffering agents. Preferably, the basic amino acid is present at from 0.1 to 2% wt. and more preferably from 0.1 to 0.5% wt. of the composition.

Suitable metal hydroxides include sodium hydroxide, potassium hydroxide and ammonium hydroxide are also useful buffering agent. Preferably, the metal hydroxide is present at from 0.001 to 2% wt. of the composition, more preferably from 0.01 to 0.5% wt. of the composition.

The composition according to the invention may comprise any of a number of other ingredients commonly found in conditioner compositions for example silicones, aminosilicones, fatty alcohols, fatty amides, fatty esters, opacifiers, perfumes, thickeners etc.

The composition may also comprise components with differing levels of lactic acid and different pH values. For example the composition may comprise a plurality of formulations which are either separated by packaging or are stored in contact with one another.

Where the composition comprises a plurality of compositions comprising different levels of lactic acid it is preferred that they are stored independent of one another to avoid transfer of lactic acid between them. Where the compositions are stored separately from one another it is preferred that they are dispensed simultaneously and are mixed immediately prior to use. Such mixing may involve nothing more than putting the compositions in contact with one another for the user to mix during use.

Where the composition has a pH of less than 3.10 it is preferred that it is in the form of a conditioning mask for intense treatment.

Preferably, the composition comprises less than 5% w/w cleansing surfactant, more preferably less than 2% wt. cleansing surfactant.

Preferably, the composition comprises less than 5% w/w anionic surfactant, more preferably less than 2% wt. anionic surfactant.

### EXAMPLE 1

The following is an example of a formulation according to an embodiment of the invention. It is made by standard processes.

The following are two conditioner compositions made by standard processes. Ingredients are shown as weight percent of total composition.

| Ingredient | A | B |
|---|---|---|
| BTAC | 1.5 | 1.25 |
| Fatty Alcohol | 6 | 5 |
| TAS | 1.5 | 1.25 |
| Lactic acid (88%) | 1.25 | 1 |
| Preservative | 0.4 | 0.4 |
| EDTA | 0.1 | 0.1 |
| NaCl | 0.1 | 0.1 |
| Silicone | 2.5 | 2.5 |
| Water | To 100 | To 100 |

In the following example, the composition is made by combining 25% v/v of Part I and 75% v/v Part II. Further, Part I and Part II may be used as conditioning treatment compositions independently of one another.

| | | | (0.25% v/v) Part I |
|---|---|---|---|
| | | | and (0.75% v/v) |
| Ingredient | Part I | Part II | Part II combined. |
| BTAC | 2 | 1 | 1.25 |
| Fatty Alcohol | 5 | 5 | 5 |
| TAS | 0 | 1.67 | 1.25 |
| Lactic Acid (88%) | 1.6 | 0.8 | 1 |
| Preservative | 0.4 | 0.4 | 0.4 |
| EDTA | 0 | 0.13 | 0.1 |
| NaCl | 0 | 0.13 | 0.1 |
| Silicone | 0 | 3.33 | 2.5 |
| Water | To 100 | To 100 | To 100 |

The following is a similar pair of conditioning formulations which may be combined to make a composition as indicated.

| | | | (0.25% v/v) Part I |
|---|---|---|---|
| | | | and (0.75% v/v) |
| Ingredient | Part I | Part II | Part II combined. |
| BTAC | 2.4 | 1.2 | 1.5 |
| Fatty Alcohol | 6 | 6 | 6 |
| TAS | 0 | 2 | 1.5 |
| Lactic Acid (88%) | 3.2 | 0.6 | 1.25 |
| Preservative | 0.4 | 0.4 | 0.4 |
| EDTA | 0 | 0.13 | 0.1 |
| NaCl | 0 | 0.13 | 0.1 |
| Silicone | 0 | 3.33 | 2.5 |
| Water | To 100 | To 100 | To 100 |

### EXAMPLE 2

The following protocol demonstrates the fibre restructuring benefit of lactic acid.

The DSC protocol measures the structure of hair. The higher the temperature the better condition the hair fibre.

The hair is first treated with a bleach. It is then treated with a conditioning composition comprising lactic acid.

Bleach Treatment

Products used:

Bleach: L'Oreal Platine Precision powder bleach.

Peroxide: 9% (30 vol) Excel crème peroxide suppliers own make. Quantity to bleach five 2.5 g/6″ switches: -

Weigh 30 g of bleach powder into a tinting bowl and 60 g of 9% Excel peroxide crème.

Mix powder and peroxide into creamy homogeneous consistency.

First Application of Bleach
1. Prepare a bleach powder/9% peroxide crème mixture as above and apply to dry hair.
2. Spread hair out into a fan on a sheet of foil.
3. Apply bleach with a tinting brush.
4. Ensure even coverage by turning the switch three times and applying the bleach crème product four times until all hair fibres are completely covered whilst in the fan-shaped conformation.
5. Bring hair fibres together and leave the switch in its normal shape to develop for 30 minutes.
6. Once the bleaching process is complete, rinse the hair switch for 2 minutes under flowing tap water. Use controlled temp/water flow-rate tap −35° C./3 to 4 l/min. The water should be clear at this stage.
7. Comb and dry the hair in a hair switch drying cabinet at 50° C. for 2 hrs.

Second Application
1. Prepare another bleach powder/9% peroxide crème mixture as above and apply to the 1× bleached dry hair.
2. Follow steps 2 to 6 as above.
3. After the bleach has been rinsed off, base wash (twice) immediately with 14% SLES 1EO+0.1% Glydant preservative (do not dry the hair prior to base washing).

Shampoo Step (1 cycle)
1. With three switches held together, pre-wet the switches under tap for ˜5 seconds at a temperature of 35-40° C. and flow rate of approx 4 litres per minute, rinse the switches under the flow of the water for 30 seconds and remove the excess water by wiping fingers along the hair (once).
2. Apply 0.25 ml of shampoo directly along the length of each switch (0.75 ml total).
3. Agitate the switches for 30 seconds while holding the ends of the switches to avoid tangling.
4. Using one of the controlled temperature 35-40° C. and flow rate of approx 4 litres per minute, rinse the switches under the flow of the water for 30 seconds. Remove the excess by running thumb and forefinger along the length of the switches.
5. Repeat the shampoo treatment steps 2-4.
NB. (a) No need to pre-wet the switches in step 2
   (b) After rinsing in step 4, remove excess water twice

Mask Step (1 cycle)

This follows immediately after the wash with shampoo.
1. Wet the switches under running water at 35-40° C. and a flow rate of approx 4 l/min.
2. Apply 0.5 g of mask to each switch.
3. With two switches held together, apply the mask to the hair and massage the switches for 1 minute, turning over the switches after ˜30 seconds and making sure to incorporate all the mask.
4. Leave for 3 minutes.
5. Using one of the controlled temperature (37° C.) and flow rate (4 litres per minute) sinks, rinse the switches under the flow of the water for 1 minute avoiding touching the switches throughout the rinse.
6. Place the switches in drying cabinet at 50° C. to dry.
7. Repeat the shampoo and mask treatment four times, which gives 5 treatments. DSC
1. One or two samples is to be taken from the middle part of a switch
2. The shavings (particles as small as possible) are collected in some aluminium foil.
3. Approximately 0.5 gram of shavings are collected per switch.
4. The DSC pan base is zeroed on the balance and then between 4-7 milligrams of the hair shavings are added to it.
   An exact record of the amount of hair shavings is kept as it is an important parameter in the DSC data analysis.
5. 50 microlitres of water are added to the hair in the DSC pan and the lid and sealing ring are put in place.
6. The sealed DSC pans is placed in a small plastic bag and agitated for 10 s on a whirly mixer.
7. At no point during this process must the pan be touched by the fingers as this could interfere with the result.

DSC Results

| | Lactic | | DSC |
|---|---|---|---|
| Arginine | Acid | pH of | Denaturation |
| wt % | wt % | Mask | Temp C. |
| 0 | 0 | 4.38 | 139.7 |
| 0.1 | 0.1 | 3.75 | 140.3 |
| 0.1 | 0.3 | 3.19 | 141.3 |
| 0.2 | 0.5 | 3.2 | 143.3 |
| 0.2 | 0.65 | 3.07 | 144.6 |
| 0.3 | 0.65 | 3.31 | 142.9 |
| 0.3 | 0.85 | 3.11 | 144.8 |
| 0.4 | 0.92 | 3.23 | 144.3 |

The results show that from above around 0.5% wt. lactic acid there is an increase in the DSC temperature which correlates with improvement in hair fibre structure.

1. Conditioning composition comprising a quaternary conditioning surfactant, an acid neutralized amidoamine surfactant of general formula:
R1-C(O)-NH-R2-N(R3)(R4)
wherein R1 is a fatty acid chain with from 12 to 22 carbon atoms, R2 is an alkylene group containing from one to 4 carbon atoms and R3 and R4 are, independently, an alkyl group having from one to four carbon atoms and from 0.45 to 4% wt. of the composition lactic acid.

2. Conditioning composition according to claim 1 wherein the pH of the composition is from 2.0 to 3.8.
